(19) United States
(12) Patent Application Publication
(10) Pub. No.: US 2023/0302242 A1
(43) Pub. Date: Sep. 28, 2023

(54) Method for the production of an assembly for a patient ventilation system

(71) Applicants: New Ventures GmbH, Rehau (DE); RAUMEDIC AG, Münchberg (DE)

(72) Inventors: Ricardo EHRENPFORDT, Zwickau (DE); Constanze RANFELD, Rehau (DE); Lydia GOLBS, Döhlau (DE); Thomas RUHLAND, Bayreuth (DE)

(73) Assignees: New Ventures GmbH, Rehau (DE); RAUMEDIC AG, Münchberg (DE)

(21) Appl. No.: 18/124,957

(22) Filed: Mar. 22, 2023

(30) Foreign Application Priority Data
Mar. 28, 2022 (DE) 10 2022 203 012.5

(51) Int. Cl. A61M 16/00

(52) U.S. Cl. CPC A61M 16/021; A61M 16/0003; A61M 2016/0018

(57) ABSTRACT

For the production of an assembly for a patient ventilation system, at least one conducting connection is first prepared between contact pins of a component receptacle of a connector for the insertion of at least one electronic component and sheath wires which are guided along a tube sheath of a breathing air tube portion of the assembly. This prepared conducting connection is overmolded to form an inner conduction carrier housing sleeve of the connector. The conducting connection is connected to the sheath wires and the inner conduction carrier housing sleeve is overmolded to form an outer connector housing of the connector in which the component receptacle is implemented. This results in an assembly manufacturing process that is suitable for mass production.

### CROSS-REFERENCES TO RELATED APPLICATIONS

This application claims the priority of German Patent Application, Serial No. DE 10 2022 203 012.5, filed Mar. 28, 2022, the content of which is incorporated herein by reference in its entirety as if fully set forth herein.

### FIELD OF THE INVENTION

The invention relates to a method for the production of an assembly for a patient ventilation system.

### BACKGROUND OF THE INVENTION

Components for a patient breathing system are known from WO 2007/051 230 A1, DE 10 2019 216 489 A1, U.S. Pat. No. 9,903,371 B2 and US 2019/0 290 866 A1. A patient breathing system “VentStar Helix” is also known from the market. DE 10 2018 218 629 A discloses a method for producing an angled connector.

### SUMMARY OF THE INVENTION

It is an object of the present invention to further develop a manufacturing method for an assembly of a patient ventilation system of this type such that it is suitable for mass production.

This object is achieved according to the invention by a method for the manufacture of an assembly for a patient ventilation system, with a breathing air tube portion for guiding breathing air from a breathing air source to a patient, wherein electrically conductive sheath wires are guided along a tube sheath of the breathing air tube portion, with a connector connected to the breathing air tube portion for connecting the breathing air tube portion to a further component of the patient ventilation system carrying the breathing air, wherein an air conduction connector component of the connector forms an integral component with the breathing air tube portion, wherein the connector comprises a component receptacle for inserting at least one electronic component into the connector, wherein the component receptacle comprises at least two contact pins for electrically connecting with the electronic component, comprising the following steps: providing at least one conducting connection between the contact pins of the component receptacle and the sheath wires, overmolding of the conducting connection to form an inner conduction carrier housing sleeve of the connector, electrically connecting the conducting connection to the sheath wires, overmolding of the inner conduction carrier housing sleeve to form an outer connector housing of the connector, in which housing the component receptacle is implemented.

According to the invention, it has been recognized that it is possible to manufacture the assembly with a manufacturing sequence that includes two overmolding steps. A first overmolding step serves to fix a prepared conducting connection, via which in particular an electrical connection between sheath wires of the breathing air tube portion and the electronic component can be established. The contact pins of the component receptacle can be designed as end portions of a respective conductor carrier of the conducting connection. The conduction carrier housing sleeve of the connector being the result of the first overmolding step comprises at least a sleeve section to guide the breathing air. In the downstream further overmolding step, a component receptacle for the electronic component is then formed together with an outer connector housing. The electronic component can then optionally be accommodated in the component receptacle or a blind cover can be inserted there. The electronic component can be a sensor device. In addition to breathing air parameters (temperature, humidity, breathing pressure, breathing air composition), the sensor device, which has at least one sensor of this type, can also be used to measure other parameters, namely parameters of the patient, those of the ventilation technique and/or parameters of the environment.

With the temperature sensor, in particular, a temperature value threshold detection is possible. This can be used for medical purposes, but also for other purposes, such as fire protection. An accuracy of the temperature sensor can be better than 0.5 K and can be, for example, 0.2 K, 0.1 K or also 0.05 K. This accuracy can be guaranteed in a range between −10° C. and 80° C. A narrower temperature range in which the accuracy is guaranteed is also possible, for example between 0° C. and 50° C. or between 10° C. and 50° C.

If a temperature sensor is used as an electronic component, it can be used, for example, to detect a temperature threshold value.

The other component of the patient ventilation system that conducts the breathing air and to which the connector of the assembly establishes a connection can be a patient interface. Alternatively, this further component can be a humidifier, for example.

A pre-shaping of the conducting connection on the basis of a 2D conductor structure which takes place during provision has been found to be particularly suitable for mass production use. The 2D conductor structure from which the conducting connection is provided may be a lead frame containing a plurality of appropriately prefabricated conducting components. The 2D conductor structure can be a stamped grid.

The 2D conductor structure can be made of metal and in particular of copper. Alternatively, the 2D conductor structure can also be a thermoset conduction carrier, for example made of PCB, and/or a conductor board.

The 2D conductor structure may have a frame carrier component that carries the respective conducting connection components. Components that are not used for a respective selected conducting connection may then be part of the frame carrier component. Such components that are not used for electrical transmission can also initially remain with the provided conducting connection as retaining components and can be removed later and, if necessary, even after electrical contacting.

The pre-shaping of the 3D conducting connection that is formed on the basis of the 2D conductor structure is performed in such a manner that the resulting conducting connection does not extend in a planar manner, but has an extension in all three spatial dimensions that exceeds the line cross-section of conducting components of the conducting connection.

An overmolding in an injection mold designed as a multiple cavity such that after pre-shaping, a plurality of conducting connections, which are associated with a corresponding plurality of inner conduction carrier housing sleeves, initially remain mechanically connected to each other, wherein during the overmolding of the conducting connections, said plurality of conducting connections are simultaneously overmolded to form the corresponding plurality of inner conduction carrier housing sleeves in a corresponding plurality of cavities of an injection mold, wherein after the overmolding for shaping the inner conduction carrier housing sleeves, a mechanical separation of the interconnected conducting connections and thus a separation of the inner conduction carrier housing sleeves takes place, increases a production throughput of the production process. The individual inner conduction carrier housing sleeves resulting from this multi-cavity overmolding can then be further processed individually and, in particular, individually further overmolded to shape the outer connector housing.

Ultrasonic welding of the electrical connection has been found to be particularly suitable for electrical connection.

An embodiment of the invention is explained in more detail below with reference to the drawing.

### BRIEF DESCRIPTION OF THE FIGURE

FIG. 1 perspectively shows an assembly for a patient ventilation system comprising a breathing air tube portion, a connector connected thereto for connecting the breathing air tube portion to a patient interface and a sensor receptacle into which a temperature sensor device is inserted;

FIG. 2 shows a side view, revealing internal details, of the assembly according to FIG. 1, again with the temperature sensor device inserted;

FIG. 3 again, perspectively shows the assembly in an intermediate stage of its production, wherein a pre-shaped 3D conducting connection is in each case integrally overmolded with contact pins of the sensor receptacle and sheath wires of the breathing air tube portion for shaping an inner conduction carrier housing sleeve of the connector;

FIG. 4 partially schematically shows processing steps in a method for the production of the assembly including the production of a plurality of inner conduction carrier housing sleeves by overmolding and subsequent mechanical separation of initially interconnected 3D conducting connections which were still connected to each other within a multiple cavity during the overmolding step;

FIG. 5 shows further processing steps of the production method up to the production of the finished assembly and a subsequent quality control;

FIG. 6 in an illustration similar to FIG. 3 showing a core of an ultrasonic welding tool, shows a further embodiment of the assembly in the intermediate stage of its production, wherein a prepared conducting connection, designed as a 2D conducting connection, is overmolded with the contact pins of the sensor receptacle and the sheath wires of the breathing air tube portion for shaping the inner conduction carrier housing sleeve of the connector;

FIG. 7 shows a top view onto the fully produced assembly, starting from the intermediate stage according to FIG. 6, wherein the assembly is shown without the inserted temperature sensor device.

### DESCRIPTION OF THE PREFERRED EMBODIMENT

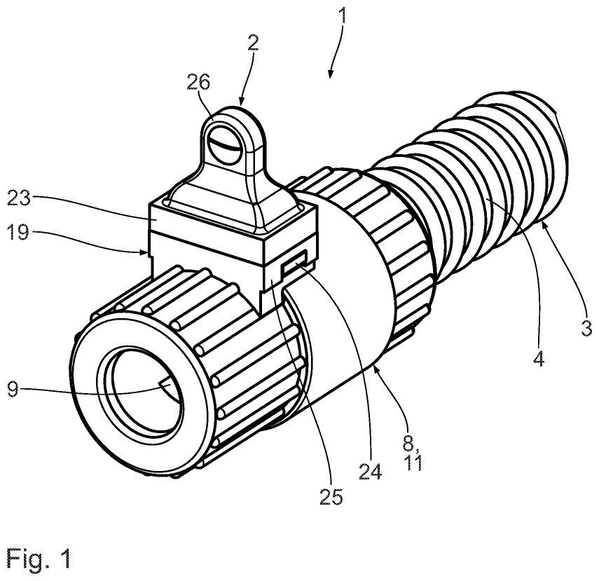

A patient ventilation system, which includes an assembly 1, which is shown in perspective in FIG. 1 with an inserted temperature sensor device 2, serves to ventilate a patient in clinical, other inpatient or also domestic care. In principle, such a patient ventilation system is known from WO 2007/051 230 A1 and DE 10 2019 216 489 A1. From the market, a corresponding patient ventilation system is known from the product “VentStar Helix”.

The main, air-conducting components of the ventilation system 1 are made of plastic.

The assembly 1 has a breathing air tube portion 3 for conducting breathing air from a breathing air source, not shown, to a patient.

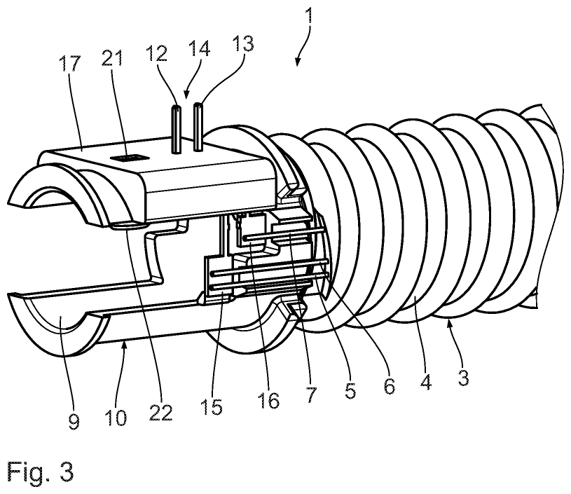

Electrically conductive sheath wires 5, 6 and 7 (cf. FIG. 3) are guided alongside a tube sheath 4 of the breathing air tube portion 3. This guidance takes place helically around an inner duct lumen of the breathing air tube portion 3. For this purpose, the sheath wires 5 to 7 are helically overmolded with material of the breathing air tube portion. Two of these sheath wires, namely the sheath wires 5 and 6 represent heating wires for the breathing air tube portion 3, which can be supplied with heating current via a supply device not shown. The third sheath wire 7 represents a signal line for the temperature sensor device 2.

A connector 8 of the assembly 1 is connected to the breathing air tube portion 3 by overmolding of the breathing air tube portion 3. The connector 8 serves to connect the breathing air tube portion 3 to a patient interface not shown in the drawing of the patient ventilation system. This connection is such that an internal air conduction connector component 9 of the connector 8 forms an integral part with the breathing air tube portion 3, at least in portions.

The connector 8 has an inner conduction carrier housing sleeve 10 and an outer connector housing 11. Those portions of the air conduction connector component 9 which are molded onto the breathing air tube portion 3 and thus form an integral component therewith are portions of the outer connector housing 11 of the connector 8.

Conducting components 12, 13 of a 3D conducting connection of the assembly 1, which 3D conducting connection extends three-dimensionally, are molded into the inner conduction carrier housing sleeve 10. This 3D conducting connection will be explained further below.

The conducting component 12 is electrically connected to the two heating sheath wires 5, 6 via a contact pad 15 and represents a short-circuit bridge for a heating circuit of the breathing air tube portion 3. The conducting component 13 is electrically connected to the signal sheath wire 7 via a contact pad 16. For contacting the contact pads 15, 16, the sheath wires 5 to 7 are exposed from the helical overmolding. Facing away from the contact pads 15, 16, the conducting components 12, 13 terminate in contact pins which protrude beyond an insulating connection plate 17 of the inner conduction carrier housing sleeve 10. When the temperature sensor device 2 is inserted, the contact pins of the conducting components 12, 13 are electrically connected to corresponding contact sockets of the temperature sensor device 2.

The two protruding contact pins of the conducting components 12, 13 serve to electrically connect the sheath wires 5 to 7 with the temperature sensor device 2.

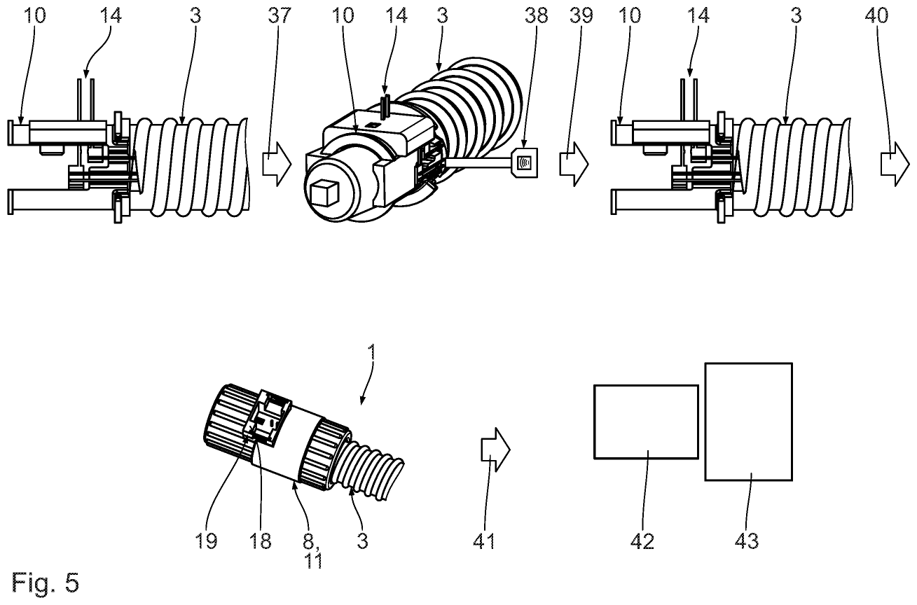

Together with a receiving body 18 (cf. the penultimate illustration of FIG. 5), which is part of the outer connector housing 11, the connecting plate 17 constitutes a sensor receptacle 19 for inserting the temperature sensor device 2 into the connector 8 and in particular for inserting a temperature sensor 20 of the temperature sensor device 2 into the air conduction connector component 9, i.e. into the air conduction lumen thereof. The sensor receptacle 19 is integrally molded into the outer connector housing 11 of the connector 8. The temperature sensor 20 serves to measure a temperature of the breathing air flowing through the air conduction connector component 9.

The temperature sensor device 2 is an example of an electronic component that can be inserted into the receptacle 19. The sensor receptacle 19 is therefore more generally a component receptacle. Instead of a temperature sensor, a sensor device for detecting another parameter, in particular a breathing air parameter, may also be used with the electronic component.

The sensor device can be a sensor for measuring air humidity or also a chemical or spectroscopic sensor. Another electronic component can also be inserted into the component receptacle 19, for example a monitoring unit, for example to determine a service life or a service load of the assembly 1.

The sensor receptacle 19 has a feedthrough 21 for passing the temperature sensor 20 of the temperature sensor device 2 into the air conduction lumen of the air conduction connector component 9. This feedthrough 21 is designed as an opening in the connection plate 17, which opens into the air conduction lumen of the air conduction connector component 9.

In addition, the sensor receptacle 19 has a sensor guiding component 22 which serves, among other things, to guide an insertion movement of the temperature sensor 20 into the air conduction lumen of the air conduction connector component 9. The sensor guiding component 22 also provides flow and/or contact protection for the temperature sensor 20.

The sensor guiding component 22 may have at least one window through which the air conduction lumen is accessible from the temperature sensor for measuring the temperature of the breathing air conducted in the air conduction lumen. The sensor guiding component 22 may contain thermally conductive components for thermally connecting the temperature sensor 20 to the air conduction lumen.

The sensor receptacle 19 is designed for plug-in insertion of the temperature sensor device 2. For this purpose, the temperature sensor device 2 has a supporting body 23 beyond which the temperature sensor 20 that is inserted into the sensor receptacle 19 protrudes downwards into the air conduction lumen of the air conduction connector component 9. A latching portion 24 (cf. FIG. 1) is formed on the supporting body 23 for latching with a counter-latching portion 25 of the sensor receptacle 19. The counter-latching portion 25 again is an integral part of the sensor receptacle 19.

Contact pins of the temperature sensor device are provided in the supporting body 23 for the connection with the contact pins of the conducting components 12, 13.

A handle portion 26 of the temperature sensor device 2 is also formed on the supporting body 23 for capturing a portion of the supporting body 23 that faces away from the temperature sensor 20.

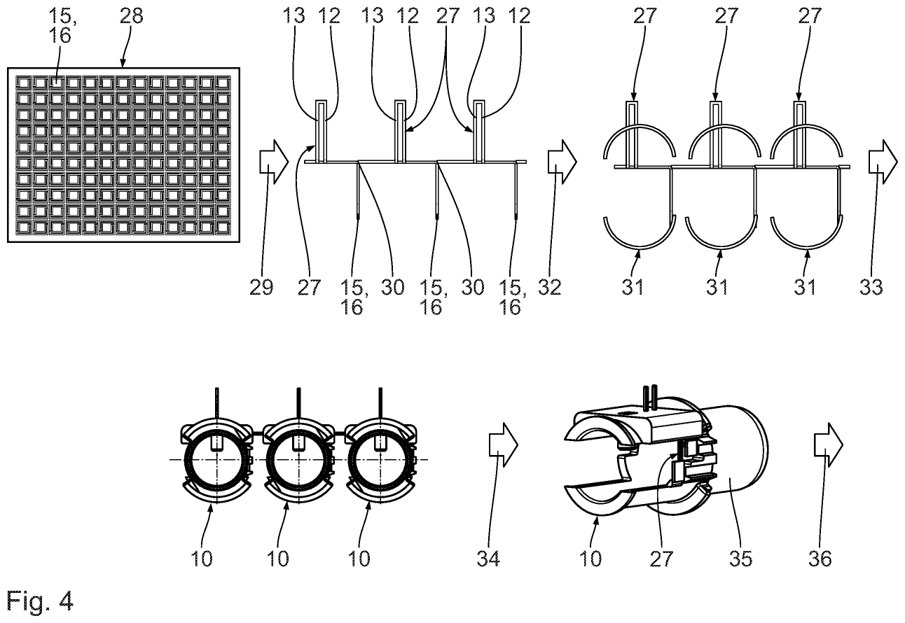

A method for the production of the assembly 1 is explained in more detail below with reference to FIGS. 4 and 5.

To produce the assembly 1, a conducting connection 27 that extends three-dimensionally is first pre-shaped on the basis of a 2D conductor structure 28 that is designed as a lead frame. The conducting components of the 2D conducting structure 28 can have cross-sections that are smaller than 0.5 mm and that can fall in the range between 50 μm and 200 μm.

The respective 3D conducting connection 27 extends between the respective contact pads 15, 16 and the contact pins of the conducting components 12, 13. In the 2D conductor structure 28, a plurality of contact pads 15 or 16 are connected to one another via corresponding conductor paths, which are bent up against one another, in particular by 90°, as part of a pre-shaping step 29 to form the 3D conducting connection 27.

The result of the pre-shaping step 29 is a plurality of 3D conducting connections 27, in the present example 3 such 3D conducting connections 27, which are assigned to a corresponding plurality of inner conduction carrier housing sleeves 10 of a respective connector 8 that are to be molded on later. After the pre-shaping step 29, this plurality of 3D conducting connections 27 are initially still mechanically connected to each other via predetermined breaking points 30.

After pre-shaping 29 of the 3D conducting connections 27, the 3D conducting connections 27, which are still mechanically connected to each other, are inserted into an injection mold in the form of an injection molding multi-cavity 31, which takes place in an insertion step 32. Subsequently, in an overmolding step 33, the 3D conducting connections 27 are overmolded to shape the inner conduction carrier housing sleeves 10 of the respective connector 8. In the overmolding step 33, the plurality of 3D conducting connections 27 that are still mechanically connected to each other are simultaneously overmolded to shape the corresponding plurality of inner conduction carrier housing sleeves 10 in the injection molding multiple cavity 31.

Result of the overmolding step 33 in particular is the forming of the inner conduction carrier housing sleeve 10 comprising at least one sleeve section serving to guide the breathing air.

After the overmolding step 33, a mechanical separation of the interconnected 3D conducting connections 27 at the predetermined breaking points takes place in a separation step 34 and thus a singulation of the inner conduction carrier housing sleeves 10.

Subsequently, a connecting sleeve 35 of the singulated inner conduction carrier housing sleeve 10 is inserted into a facing end portion of the breathing air tube portion 3 in an insertion step 36.

FIG. 5 shows on the upper left a corresponding inner conduction carrier housing sleeve 10 inserted into the breathing air tube portion 3. In the course of the insertion step 36, the contact pads 15, 16 of the conducting components 12, 13 are positioned and aligned towards the exposed end portions of the sheath wires 5 to 7.

In a following connection step 37, the conducting components 12, 13 of the conducting connection 14 or 27 are electrically contacted with the end portions of the sheath wires 5 to 7. This is performed by means of ultrasonic welding with a corresponding ultrasonic welding device 38.

Afterwards, in an insertion step 39, the prefabricated raw assembly with the inner conduction carrier housing sleeve 10 and the breathing air tube portion 3 that is mechanically and electrically connected thereto is inserted into another injection mold. In a subsequent further overmolding step 40, the inner conduction carrier housing sleeve 10 and an end region of the breathing air tube portion 3 adjacent thereto are then overmolded to shape the outer connector housing 11. The sensor receptacle 19 is also shaped in the outer connector housing during this process. Via the overmolding in the further overmolding step 40 an integration of the air conduction connector component 9 with the breathing air tube portion 3 results. Due to this, the air conduction connector component 9 of the produced connector 8 forms an integral part with the breathing air tube portion.

The electronic component which can be accommodated in the component or sensor receptacle 19 can generally be a sensor device, for example the temperature sensor device 2.

After the further overmolding step 40, an optical quality control 42 and an electrical quality control 43 are carried out in a control step 41, wherein the assembly 1 can be subject to a visual inspection as well as an inspection by corresponding optical and/or electrical/electronic measuring units.

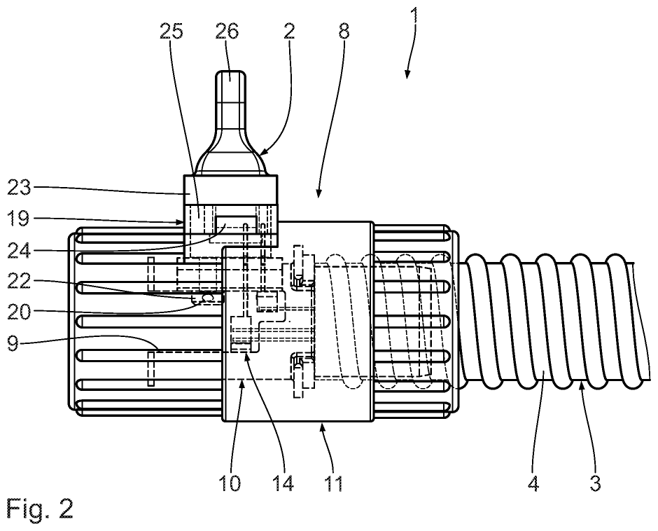

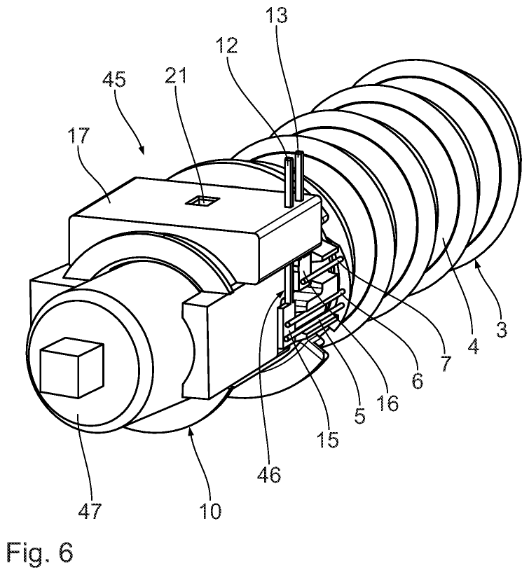

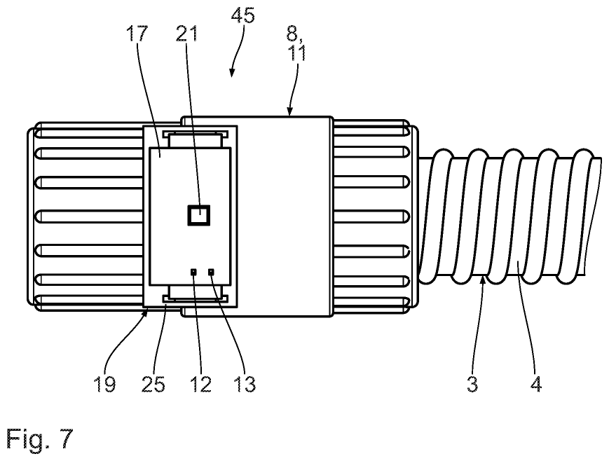

With reference to FIGS. 6 and 7, a further embodiment 45 of the assembly for the patient ventilation system is described below, which can be used instead of the assembly described above with reference to FIGS. 1 to 5.

Components and functions that correspond to those already explained above with reference to FIGS. 1 to 5 bear the same reference numerals and will not be discussed again in detail.

In the assembly 45, a conducting connection 46, which otherwise corresponds to the 3D conducting connection 27 of the embodiment according to FIGS. 1 to 5, is designed in two dimensions. The contact pins 12, 13 of this conducting connection and the contact pads 15, 16 are located in a common arrangement plane of the 2D conducting connection 46.

FIG. 6 also shows a core 47 of an ultrasonic welding tool for ultrasonic welding of the conducting components 12, 13 of the conducting connection 14 and 27, respectively, to the end portions of the sheath wires 5 to 7 of the assembly 45.

The production method of the assembly 45 is basically the same as the one already explained above with reference to FIGS. 4 and 5.

Instead of pre-shaping a 3D conducting connection, the 2D conducting connection 46 is first provided during the production of the assembly 45, on the basis of the 2D conductor structure 28 that is designed as a lead frame. This provision can take place exclusively by separating the associated conducting components from the surrounding carrier or conducting components of the lead frame 28.

The result of this provision of the 2D conducting connections 46 is a plurality of such 2D conducting connections 46, for example three 2D conducting connections 46 of this type, which are assigned to a corresponding plurality of the inner conduction carrier housing sleeves 10, which are to be molded on later, of the respective connector 8 of the assembly 45. This plurality of 2D conducting connections 46 are initially still mechanically connected to one another via predetermined breaking points after the provision step, as already explained above with reference to the 3D conducting connections 27 of the embodiment according to FIGS. 1 to 4.

Then, during the production of the assembly 45, the insertion 32, the overmolding, the separation 34, the insertion of the connection sleeve 35 including the positioning and alignment of the contact pads 15, 16 to the provided end portions of the sheath wires 5 to 7, the electrical connection 37, the insertion 39, the further overmolding 40 as well as the control 41 are carried out according to what has already been explained above in connection with the production of the assembly 1 according to FIGS. 1 to 5.

What is claimed is:

1. A method for the manufacture of an assembly (1; 45) for a patient ventilation system,
with a breathing air tube portion (3) for guiding breathing air from a breathing air source to a patient, wherein electrically conductive sheath wires (5 to 7) are guided along a tube sheath (4) of the breathing air tube portion (3),
with a connector (8) connected to the breathing air tube portion (3) for connecting the breathing air tube portion (3) to a further component of the patient ventilation system carrying the breathing air, wherein an air conduction connector component (9) of the connector (8) forms an integral component with the breathing air tube portion (3),
wherein the connector (8) comprises a component receptacle (19) for inserting at least one electronic component (2) into the connector (8),
wherein the component receptacle (19) comprises at least two contact pins (12, 13) for electrically connecting with the electronic component (2),
comprising the following steps:
providing at least one conducting connection (27) between the contact pins (12, 13) of the component receptacle (19) and the sheath wires (5 to 7),
overmolding (33) of the conducting connection (27) to form an inner conduction carrier housing sleeve (10) of the connector (8),
electrically connecting (37) the conducting connection (27) to the sheath wires (5 to 7),
overmolding (40) of the inner conduction carrier housing sleeve (10) to form an outer connector housing (11) of the connector (8), in which housing the component receptacle (19) is implemented.

2. The method according to claim 1, wherein pre-shaping (29) of the conducting connection on the basis of a 2D conductor structure (28) takes place during provision.

3. The method according to claim 2, wherein during pre-shaping (29) a 3D conducting connection is formed on the basis of the 2D conductor structure (28).

4. The method according to claim 1, wherein, after pre-shaping (29), a plurality of conducting connections (27), which are associated with a corresponding plurality of inner conduction carrier housing sleeves (10), initially remain mechanically connected to each other,
wherein during the overmolding (33) of the conducting connections (27), said plurality of conducting connections (27) are simultaneously overmolded to form the corresponding plurality of inner conduction carrier housing sleeves (10) in a corresponding plurality of cavities of an injection mold (31),
wherein after the overmolding (33) for shaping the inner conduction carrier housing sleeves (10), a mechanical separation of the interconnected conducting connections (27) and thus a separation of the inner conduction carrier housing sleeves (10) takes place.

5. The method according to claim 1, wherein the electrical connection (37) is performed by ultrasonic welding.
